(19) United States
(12) Patent Application Publication
(10) Pub. No.: US 2008/0260633 A1
(43) Pub. Date: Oct. 23, 2008

(54) SUPRAMOLECULAR COMPLEXES AS PHOTOCATALYSTS FOR REDUCTION OF SUBSTRATES

(76) Inventors: Karen J. BREWER, Blacksburg, VA (US); Ran Miao, Blacksburg, VA (US)

Correspondence Address: BLANK ROME LLP, 600 NEW HAMPSHIRE AVENUE, N.W., WASHINGTON, DC, 20037, US

(21) Appl. No.: 12/052,955

(22) Filed: Mar. 21, 2008

Related U.S. Application Data
(60) Provisional application No. 60/896,103, filed on Mar. 21, 2007.

(51) Int. Cl. C01B 3/08

(52) U.S. Cl. 423/657

(57) ABSTRACT

Supramolecular complexes, methods and systems for photocatalysis of the reduction of substrates are described. The supramolecular complexes of the invention have a light absorbing metal center, an electron collector ligand and a catalytically active metal. When the supramolecular complexes are exposed to radiant energy, the light absorbing metal center creates a charge that is transferred to the electron collector ligand to form a charge transfer state. The charge is then transferred through the catalytically active metal to a substrate, cause the reduction of the substrate, e.g. the reduction of water to molecular hydrogen.

### STATEMENT OF PRIORITY

This application claims priority to U.S. Provisional Patent Application Ser. No. 60/896,103, filed Mar. 21, 2007, which is hereby incorporated by reference.

### FIELD OF THE INVENTION

The present invention relates generally to supramolecular complexes designed to convert light energy into chemical energy. More specifically, the present invention relates to supramolecular complexes having a light absorbing metal center capable of capturing light energy and transferring it to a catalytically active metal center capable of catalyzing a chemical reaction.

### BACKGROUND

The efficient and environmentally friendly production of hydrogen is one of the keys to the “hydrogen economy” or use of hydrogen as an alternative to petroleum based fuels. The need for an alternative and better methods for producing hydrogen drives light to energy conversion research aimed at solar energy conversion schemes.^{1,2,3} The discovery of [Ru(bpy)₃]²⁺ (bpy=2,2′-bipyridine) led to photochemical studies exploring the use of metal to ligand charge transfer (MLCT) states in light to energy conversion. [Ru(bpy)₃]²⁺ and related complexes possess ³MLCT excited states of sufficient energy to drive water splitting to H₂ and O₂. Most systems reported to date that use Ru light absorbers to produce H₂ use electron relays and heterogeneous metal catalysts.⁴ Upon MLCT excitation of [Ru(bpy)₃]²⁺, bimolecular electron transfer to [Rh(bpy)₃]³⁺ occurs generating [Rh(bpy)₃]²⁺. In the presence of H₂O and a heterogeneous platinum catalyst, this system generates H₂.^{4d} Recently Eisenberg et at reported a colloidal platinum terpyridine chromophore that photocatalyzes hydrogen production from water utilizing a metallic Pt catalyst.⁵ Nocera et al. reported a homogenous dirhodium system that photocatalyzes hydrogen production from hydrohalic acids with a 0.01 quantum efficiency.⁶

Much research has focused on the development of Ru polymetallic polyazine complexes.⁷ Fewer systems have been reported coupling Ru light absorbers (LAs) to Pt through polyazine bridging ligands. Complexes such as [(tpy)RuCl(dpp)PtCl₂]⁺ (tpy=2,2′:6′,2″-terpyridine) are reported to bind DNA.⁸ The [(bpy)₂Ru(dpp)PtCl₂]²⁺, [(bpy)₂Ru(dpq)PtCl₂]²⁺ and [(bpy)₂Ru(dpb)PtCl₂]²⁺ have been reported (dpb=2,3-bis(2-pyridyl)benzoquinoxaline).¹⁰ Recently, the inventors reported [{(bpy)₂Ru(dpp)}₂Ru(dpp)PtCl₂](PF₆)₆ as a multifunctional DNA binding, photocleavage agent.¹¹ Mixed-metal polyazine complexes have been recently explored, which couple ruthenium LAs to reactive metal sites. Sakai recently investigated a Ru—Pt bimetallic system capable of photochemically producing hydrogen from water in the presence of the electron donor, ethylenediaminetetraacetic acid (EDTA)¹² functioning with Φ≈0.01, and 5 turnovers in 10 h. This low turnover has since been postulated to reflect decomplexation of the reactive metal to form colloidal platinum, which functions as the hydrogen generation site. Rau reported a Ru—Pd bimetallic system that photochemically produces hydrogen in the presence of the electron donor, TEA.¹³ This system was shown to produce colloidial Pd.¹⁴

In general, the production of H₂ from H₂0 requires the catalysis of a multielectron reduction. To catalyze a multielectron reduction, a photocatalysts must be able to collect electrons until sufficient electrons are accumulated to cause catalysis. Many of the known photocatalysts have electron collecting metal centers, such as those described by Brewer et al. in U.S. Patent Application Publication 2006/0286027, which is hereby incorporated by reference.

Despite advances in the field, there remains a need in the art for efficient, stable supramolecular complexes that are capable of turning light energy into chemical energy for catalyzing H₂ production from H₂O, along with other chemical reactions.

### SUMMARY OF THE INVENTION

It is an object of the present invention to provide supramolecular complexes capable of converting radiant energy into chemical energy. The supramolecular complexes of the present invention are photocatalysts capable of harvesting radiant energy and using it for catalyzing chemical reactions. The supramolecular complexes are molecular assemblies having a light absorber, a bridging ligand, electron collector and catalytically active metal center. This type of supramolecular complex is a photochemical molecular device, and is designed so that modification of the device components allows for the modification and optimization of the functioning of the molecular device.

It is another object of the present invention to provide supramolecular complexes capable of catalyzing chemical reactions with high efficiency and stability. The supramolecular complexes of the present invention are designed to provide high turnover for extended periods of time.

It is a further object of the present invention to provide a method for photocatalytic reduction of a substrate. The method involves contacting the substrate with a supramolecular complex capable of photocatalyzing the reduction of the substrate, followed by exposing the supramolecular complex to a source of radiant energy.

It is a still further object of the present invention to provide a system for the photocatalytic reduction of a substrate. The system of the present invention has a vessel for containing the substrate and a supramolecular complex capable of photocatalyzing the reduction of the substrate. The system of the present invention also has a source of radiant energy which can be used to cause the catalysis.

### BRIEF DESCRIPTION OF THE DRAWINGS

FIG. 1 shows the molecular structure of exemplary terminal and bridging ligands.

FIG. 2 is a schematic representation of an embodiment of a system of the present invention for photocatalysis of the reduction of a substrate.

FIG. 3 shows a synthetic route for preparing the tetrametallic [{(bpy)₂Ru(dpp)}₂Ru(dpp)PtCl₂](PF₆)₆ where bpy=2,2′-bipyridine, dpp=2,3-bis(2-pyridyl)pyrazine. a. ref²⁹, b. ref.³⁰, c. ref²³, d. ref³¹.

FIG. 4 is a cyclic voltammetry plot of [{(bpy)₂Ru(dpp)}₂Ru(dpq)](PF₆)₆ measured in 0.1 M Bu₄NPF₆ CH₃CN solution at room temperature at a scan rate 200 mV/s, potential reported in V vs Ag/AgCl (bpy=2,2′-bipyridine, dpp=2,3-bis(2-pyridyl)pyrazine, dpq=2,3-bis(2-pyridyl)quinoxaline).

FIG. 5 is a square wave voltammetry plot of [{(bpy)₂Ru(dpp)}₂Ru(dpq)](PF₆)₆ measured in 0.1 Bu₄NPF₆ CH₃CN solution at room temperature at a scan rate 200 mV/s, potential reported in V vs Ag/AgCl (bpy=2,2′-bipyridine, dpp=2,3-bis(2-pyridyl)pyrazine, dpq=2,3-bis(2-pyridyl)quinoxaline).

FIG. 6 is a cyclic voltammetry plot of [{(bpy)₂Ru(dpp)}₂Ru(dpq)PtCl₂](PF₆)₆ measured in 0.1 M Bu₄NPF₆ CH₃CN solution at room temperature at a scan rate 200 mV/s, potential reported in V vs Ag/AgCl (bpy=2,2′-bipyridine, dpp=2,3-bis(2-pyridyl)pyrazine, dpq=2,3-bis(2-pyridyl)quinoxaline).

FIG. 7 is a square wave voltammetry plot of [{(bpy)₂Ru(dpp)}₂Ru(dpq)PtCl₂](PF₆)₆ measured in 0.1 M Bu₄NPF₆ CH₃CN solution at room temperature at a scan rate 200 mV/s, potential reported in V vs Ag/AgCl (bpy=2,2′-bipyridine, dpp=2,3-bis(2-pyridyl)pyrazine, dpq=2,3-bis(2-pyridyl)quinoxaline).

FIG. 8 shows the electronic absorption spectrum of [{(bpy)₂Ru(dpp)}₂Ru(dpq)PtCl₂](PF₆)₆ in CH₃CN at RT (bpy=2,2′-bipyridine, dpp=2,3-bis(2-pyridyl)pyrazine, dpq=2,3-bis(2-pyridyl)quinoxaline).

FIG. 9 shows a state Diagram for [{(bpy)₂Ru(dpp)}₂Ru(dpq)PtCl₂](PF₆)₆ in CH₃CN at RT (bpy=2,2′bipyridine, dpp=2,3-bis(2-pyridyl)pyrazine, dpq=2,3-bis(2-pyridyl)quinoxaline).

FIG. 10 shows electronic absorption spectra for the electrochemical reduction (A) and photoreduction (B) of [{(bpy)₂Ru(dpp)}₂Ru(dpq)PtCl₂](PF₆)₆ in RT CH₃CN. Spectroelectrochemistry in 0.1 M Bu₄NPF₆ solution with a platinum mesh working electrode. Spectrum 2A: Initial (—), −0.20 V (---), −0.55 V ( . . . ). Photolyses were carried out with 5 mM dimethylaniline and irradiated with a 455 nm LED Spectrum 2B: Initial (—), after photolysis ( . . . ). (bpy=2,2′-bipyridine, dpp=2,3-bis(2-pyridyl)pyrazine, dpq=2,3-bis(2-pyridyl)quinoxaline).

FIG. 11 shows a comparison of electronic absorption spectra prior to (—) and after (--) the photolysis for [{(bpy)₂Ru(dpp)}₂Ru(dpq)PtCl₂](PF₆)₆.

FIG. 12 shows a plot of Hydrogen generated amount vs. photolysis time for tetrametallic complexes [{(bpy)₂Ru(dpp)}₂Ru(dpq)PtCl₂](PF₆)₆. (bpy=2,2′bipyridine, dpp=2,3-bis(2-pyridyl)pyrazine, dpq=2,3-bis(2-pyridyl)quinoxaline).

FIG. 13 shows a Stem-Volmer plot for a DMA quenching experiment on tetrametallic complexes [{(bpy)₂Ru(dpp)}₂Ru(dpq)PtCl₂] (PF₆)₆ in CH₃CN.

### DETAILED DESCRIPTION OF THE INVENTION

The present invention provides supramolecular systems or complexes capable of undergoing photoinitiated multi-electron processes. The supramolecular complexes are capable of photoinitiated electron collection. The collected electrons can be used to drive multi-electron reactions. The supramolecular complexes of the present invention allow electrons to be collected on an electron collector of the supramolecular assembly and used to catalyze a chemical reaction while the complex remains intact following catalysis.

The supramolecular complexes of the present invention also possess the ability to be further reduced on the π* system other bridging ligands. This ability to undergo further photoreduction provides additional electrons or electrons with a higher potential available for the reduction of a desired substrate.

The supramolecular complexes of the present invention may include one or more light absorber, bridging ligand, electron collector, and catalytically active metal center. In the supramolecular complex, a light absorber is chemically coupled to an electron collector via one or more bridging ligand, which serves as a route for the transmission of electrons. The light absorber may also be coordinated in supramolecular complexes by one or more terminal ligand. In certain embodiments of the present invention, the bridging ligands and terminal ligands may be the same type of molecule.

In the supramolecular complexes of the present invention, the following essential components are coupled: 1) at least one light absorbing metal center, 2) an electron collecting ligand or set of ligands, and 3) a catalytically active metal. The metal to ligand charge transfer light absorber produces an initially optically populated metal to ligand charge transfer state. The electron acceptor ligand, which may be a single bridging ligand or a series of ligands coordinated to a single metal center possesses a π system capable of being involved in an initial metal to ligand charge transfer (MLCT) excitation, followed by the formation of a charge separation (CS) state. The catalytically active metal is then capable of promoting transfer of the electrons localized on the electron acceptor ligand to a substrate, facilitating a chemical reaction.

The supramolecular complexes of the present invention are useful for catalyzing multi-electron reduction reactions in that they are able to accumulate electrons on an electron collector ligand and maintain these accumulated electrons in a charge separated state until a sufficient number of electrons is accumulated to catalyze the reaction desired. When the supramolecular complexes of the present invention are exposed to radiation (e.g. visible or ultraviolet light) the light absorbing metal centers cause the movement of electrons to an electron collector ligand, where they are maintained until a sufficient number of electrons is accumulated to catalyze the desired reduction. Once a sufficient number of electrons is accumulated on the electron collector ligand, the accumulated charge is then transferred through the catalytically active metal to the substrate, causing the multi-electron reduction of the substrate.

The number and type of MLCT light absorbers used in the supramolecular metallic complexes of the present invention may vary, depending on several factors including but not limited to: the desired excitation wavelength to be employed; the oxidation potential of interest for the metal based highest occupied molecular orbital; the required extinction coefficient for the excitation wavelength; ease of synthesis of the complex; cost and/or availability of components; and the like. Any suitable number of MLCT light absorbers may be used so long as an initial optically populated MLCT state is produced within the complex upon exposure to light or radiant energy, which can be relayed to a suitable electron collector ligand. In certain embodiments of the present invention, the number of MLCT light absorbers will range from 1 to about 14.

Those of skill in the art will recognize that many suitable metals exist that can function as MLCT light absorbers in the practice of the present invention. Examples include but are not limited to ruthenium(II), osmium(II), rhenium (I), iron(II), and the like. In certain embodiments of the present invention, ruthenium(II) or osmium(II) centers are utilized. Further, it is contemplated that more than one type of light absorber may be utilized in one supramolecular complex.

The complexes of the present invention typically have at least one bridging π-acceptor ligand capable of being involved in an initial metal to ligand charge transfer excitation. A bridging ligand in the context of the present invention is a π-acceptor ligand that, in the supramolecular complex, may be located or positioned (e.g. bonded, coordinated) between MLCT light absorbers or between an MLCT light absorber and the catalytic metal center. As will be described below, the bridging ligand may also function as an electron collector ligand. The number of bridging ligands in a supramolecular complex varies depending on the number of MLCT light absorbers and electron collector ligands in the complex. In certain embodiments, the number will range from about 1 to about 14 or more.

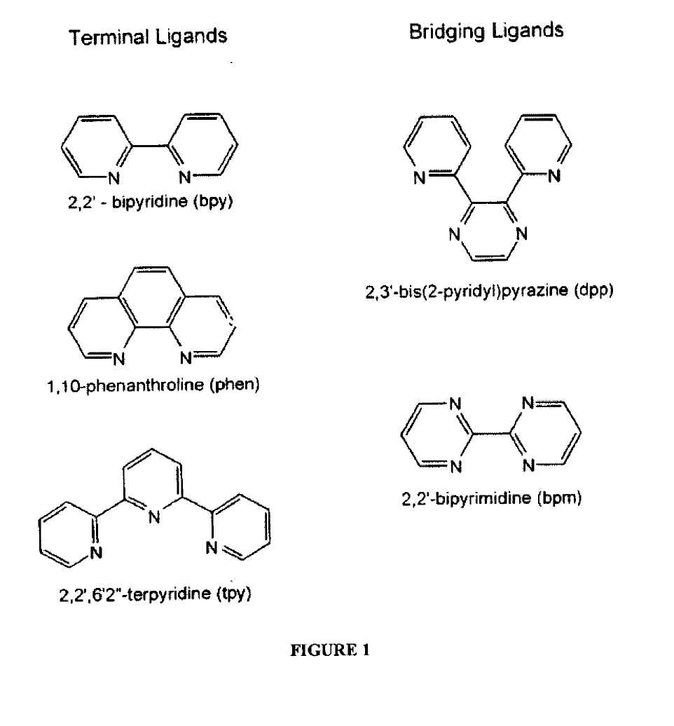

The bridging π-acceptor ligands coordinate or bind to the metal centers via donor atoms and bridging ligands have the ability to bind two or more metal centers. Those of skill in the art will recognize that many suitable substances exist which contain appropriate donor atoms and may thus function as π-acceptor ligands in the complexes of the present invention, generally identified as Lewis bases or ligands. Examples include but are not limited to substances with: nitrogen donor atoms (e.g. pyridine-, pyrazine- and pyridimidine-containing moieties such as 4,4′-bipyridine (“bpy”); 2,2′:6′,2″-terpyridine (“tpy”); 2,3-bis(2-pyridyl)pyrazine (“dpp”); and 2,2′-bipyridimidine (“bpm”); 2,3-bis(2-pyridyl)quinoxaline; 2,3,5,6,-tetrakis(2-pyridyl)pyrazine; carbon and nitrogen donor atoms (e.g. 2,3-diphenylpyridine); phosphorus donor atoms (e.g. diethylphosphinoethane); and the like. Some non-limiting examples of bridging ligands are shown in FIG. 1.

The supramolecular complexes of the present invention may also include terminal ligands. Terminal ligands bind or coordinate to only one metal center and serve to satisfy the needed coordination sphere for the metals in the supramolecular complex, thereby providing a means to tune both light absorbing and redox properties of the metal centers. Many suitable substances exist which can function as terminal ligands, many of which may also be used as bridging ligands. For example, substances with: nitrogen donor atoms (e.g. pyridine-, pyrazine and pyridimidine-containing moieties such as 2,2′-bipyridine (“bpy”); 2,2′:6′,2″-terpyridine (“tpy”); 2,3-bis(2-pyridyl)pyrazine (“dpp”); and 2,2′-bipyridimidine (“bpm”); 2,3-bis(2-pyridyl)quinoxaline; 2,3,5,6,-tetrakis(2-pyridyl)pyrazine; carbon and nitrogen donor atoms (e.g. 2,2′-phenylpyridine); phosphorus donor atoms (e.g. triphenylphosphine, diethylphenylphosphine); and the like. It is also contemplated that the terminal ligands may be other ligands that are well know in the art, including halides (e.g. Cl⁻, Br⁻, I⁻, F⁻); phosphines having the general formula PR₃ (where each R may be the same or different and may be alkyl or aryl groups having between 1 and 12 carbons); simple amines having a general formula NR₃ (where each R may be the same or different and may be hydrogen or alkyl or aryl groups having between 1 and 12 carbons); CN, CO; COOH; H₂O; CH₃CN; pyridines; hydrides; and the like. Some non-limiting examples of bridging ligands are shown in FIG. 1.

In the supramolecular complexes of the present invention, the bridging and terminal ligands may be the same type of molecule. In addition, two or more different types of molecules may function as terminal ligands in a supramolecular complex.

The electron collector ligand or set of ligands of the present invention may be either a bridging ligand, a terminal ligand, or combinations thereof. The electron collector ligand must be able to not only receive electrons from the excited state of the MLCT light absorbers, but must also be able to collect reducing equivalents. In certain embodiments, the electron collector ligand is dpq, dpb, dpp or tppz. It is also contemplated that other ligands can be used as an electron collector ligand, including those listed above as bridging and terminal ligands as well as other ligands know in the art which are known to act as electron collectors. It should be apparent that one or more than one electron collector ligand may be present in the supramolecular complexes of the present invention, and that there may be different types of electron collector ligand in a single supramolecular complex.

The catalytically active metal is used to facilitate electron transfer from the electron collector to the substrate. Those of skill in the art will recognize that many metals may be used as catalytically active metals in the complexes of the present invention. Examples of suitable metals include but are not limited to platinum (II), palladium (II), cobalt (I), rhodium (I) and iridium (I). Any metal that can bind to an electron collector ligand and deliver the collected electrons to a substrate may be utilized. In certain embodiments of the present invention, the catalytically active metal is platinum (II).

It is also contemplated that the number of catalytically active metals in the complex may be varied. Multifunctional systems could be designed that use many electron collector ligand sites coupled with a catalytically active metal to enhance the functioning of the system by providing additional active sites within a single molecular architecture. Importantly, the design of the supramolecular complexes of the present invention is such that the complex remains intact and is not destroyed upon carrying out catalytic functions. The advantage of this attribute is that systems employing the supramolecular complexes of the present invention are capable of carrying out repeated catalytic reactions when coupled to electron donors or water oxidation and are thus long-lived in comparison to known systems. The light absorbers remain attached to the electron collector following electron collection, which allows the complexes to undergo further charge transfer excitation, leading to there catalytic activity.

In general, the supramolecular architecture of the complexes of the present invention can be varied by changing the identity and number of components of the complex. However, it is necessary to retain the components in sufficiently close association and appropriate orientation to provide the necessary electronic coupling. This coupling is necessary to allow electron transfer from the MLCT light absorber to the electron collecting ligand and then to the catalytically active metal. Those of skill in the art will recognize that the precise distances between components and the orientation of the components will vary from complex to complex, depending on the identity of complex substituents. However, in general the distances will be confined to the multi-atomic or multi-angstrom scale.

Those of skill in the art will recognize that many such suitable counterions exist and may be utilized to form the salt form of a complex without altering the fundamental properties of the complex, other than its solubility.

Synthesis of the supramolecular complexes of the present invention can be carried out by a building block approach. A non-limiting example of this type of synthesis is shown the Examples below. Generally using this method the terminal metals on the outside of the complex are prepared first, reacting them with the desired terminal ligands. Once the terminal metal is coordinated to the terminal ligand, reaction with a bridging ligand assembles that sub-unit of the supramolecular complex. These sub-units are then reacted with additional metals, either secondary light absorbing units or the reactive metal. This means of assembly allows for control of supramolecular structure.

Alternatively, the supramolecular complexes can be assembled from the center out by reacting the desired bridging ligands with the reactive metal followed by coupling to the light absorbing units.

Suitable wavelengths of light for use in the practice of the present invention are dependent on the components of a given supramolecular complex. In general, visible (e.g. light with a wavelength greater than 400 nm) and ultraviolet light can be utilized. In general, the wavelength used will depend on the supramolecular complex of interest and its ability to absorb at that wavelength. Typically excitation will occur in the region of the intense metal to ligand charge transfer excitation. However, those of skill in the art will recognize that other excitations further from the optimum can also be used due to the efficient internal conversion within supramolecular complexes of the type described herein. Suitable sources of excitation include various well-known artificial sources and natural sunlight.

The supramolecular complexes of the present invention may catalyze the reduction of a variety of substrates, particularly substrates that are suitable for multi-electron reduction. In one embodiment of the present invention, the supramolecular complexes catalyze the reduction of water to H₂. In other embodiments, the substrate for reduction may be carbon dioxide, carbon monoxide, methanol and nitrobenzene.

The present invention also provides a method of reducing a substrate. The method of the present invention involves exposing the substrate to a supramolecular complex of the invention and to radiant energy. Generally, the supramolecular complex is brought into contact with the substrate, and is then exposed to a source of radiant energy having a wavelength which causes the supramolecular catalyst to catalyze the reduction of the substrate. In certain embodiments of the invention, the substrate itself functions as the electron donor for the reduction. However, it is also contemplated that other electron donors may also be used in the methods of the present invention, including but not limited to dimethylaniline (DMA), triethanolamine (TEOA), triethylamine (TEA), ascorbic acid and the like.

The present invention also provides a method of producing molecular hydrogen from water. In these embodiments of the invention, water is reduced to molecular hydrogen by exposing the water to a supramolecular complex of the invention and to radiant energy, in a manner analogous to the general method described above. In certain embodiments of the invention, the water itself functions as the electron donor for the reduction. However, it is also contemplated that other electron donors may also be used in reducing water to molecular hydrogen, including but not limited to dimethylaniline (DMA), triethanolamine (TEOA), triethylamine (TEA), ascorbic acid and the like.

The invention further provides a system/apparatus for catalyzing the reduction of water to form hydrogen which incorporates these materials either photochemically or electrochemically. Amouyal and Sauvage have highlighted systems that photochemically produce hydrogen from water in separate reviews.^{15,16} A system to produce hydrogen from water using light would include the supramolecular complexes as described herein and additional components that would be involved in the electron donation and the use of the oxidizing equivalents to oxidize a substrate, for example the oxidation of water to oxygen. The general design of such systems is known to those of skill in the art, and can readily be adapted to include the supramolecular complexes of the present invention. The supramolecular complexes will perform the function of light absorber, electron collector and catalyst for the conversion of light energy into chemical energy. The supramolecular complexes could be in solution or attached to a support, depending on whether homogeneous or heterogeneous catalysis is desired.

In a preferred embodiment of the invention, the system is coupled to a water oxidation cycle that produces oxygen and allows water to function as the electron donor. However, those of skill in the art will recognize that other electron donors may also be used in the methods of the present invention, including but not limited to dimethylaniline (DMA), triethanolamine (TEOA), triethylamine (TEA), EDTA, and ascorbic acid.

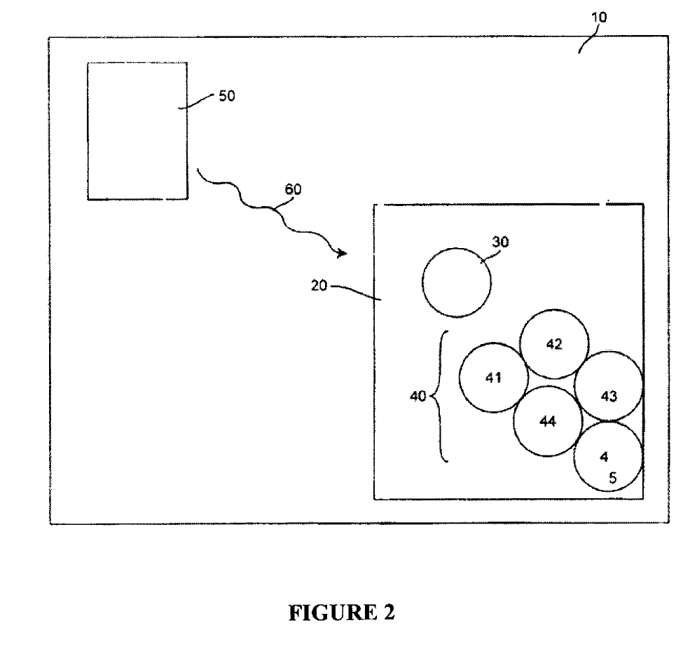

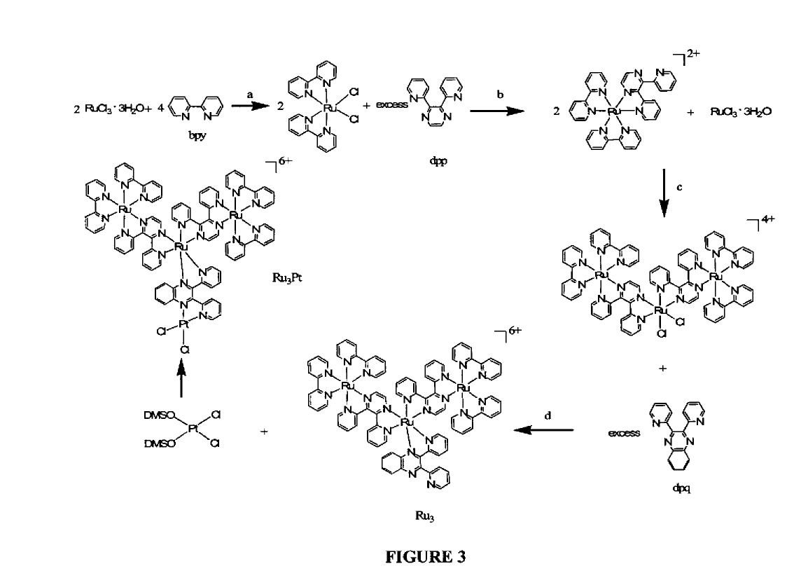

FIG. 2 illustrates an exemplary system for reducing water to produce hydrogen. As illustrated in the Figure, the system 10 comprises a vessel 20 for containing water 30 and a supramolecular complex 40. The supramolecular complex 40 comprises at least one metal to ligand charge transfer light absorbing metal 41, at least one electron acceptor ligand 42; at least one catalytically active metal 43; at least one terminal ligand 44; and an electron donor 45 which interacts with the supramolecular complex. Although not specifically shown in FIG. 2, it is also contemplated that electron donor 45 can be separate from the supramolecular complex, e.g., not physically interacting with the supramolecular complex. The system further comprises means 50 for directing radiant energy 60 towards the vessel.

The system of the present invention may be coupled to an oxidation process such as a water to oxygen oxidation or another scheme that uses oxidizing equivalents. In one embodiment of a system of the present invention, a sacrificial electron donor is utilized. The donor can then be used as an oxidizing equivalent relay to couple to a complementary oxidation process. In certain embodiments, the oxidation chemistry is not photochemical but rather simple redox chemistry. For example, in a solar cell that uses light energy to split water, the light energy excites electrons at a collection site that then reacts with water to produce hydrogen. The oxidizing equivalents move to the positive electrode and are collected at a catalyst that can oxidize water to oxygen to complete a catalytic cycle.

In other embodiments, it is also contemplated that the system of the present invention may be modified for photocatalysis of other substrates. In such modifications, the substrate to be reduced is placed in the vessel in contact with a supramolecular complex capable of catalyzing the reduction of the substrate. Electron donors may be added to the vessel as described above.

The following examples are to be considered as exemplary of various aspects of the present invention and are not intended to be limiting with respect to the practice of the invention. Those of ordinary skill in the art will appreciate that alternative materials, conditions, and procedures may be varied and remain within the skill of the ordinarily skilled artisan without departing from the general scope of the invention as claimed below.

### EXAMPLES

### Example 1

### Synthesis of [{(bpy)₂Ru(dpp)}₂Ru(dpq)PtCl₂](PF₆)₆

The title complex [{(bpy)₂Ru(dpp)}₂Ru(dpq)PtCl₂](PF₆)₆ is prepared via a building block method coupling terminal (bpy)₂Ru^{II}(dpp) LA units to the central Ru²⁵ followed by addition of dpq, then complexation of Pt^{II}Cl₂.

Materials—Ruthenium(III) chloride hydrate (Alfa Aesar), platinum colloid (polyethyleneglycoldodecylether hydrosol) (Strem), previously-reported dpq (2,3-bis(2-pyridyl)quinoxaline)^{17,18} [PtCl₂(DMSO)₂] (DMSO=dimethylsulfoxide)¹⁹, 2,2′-bipyridine (bpy) (Sigma-Aldrich), 2,3-bis(2-pyridyl)pyrazine (dpp) (Aldrich), (80-200 mesh) adsorption alumina (Fisher), tetrabutylammonium hexafluorophosphate Bu₄NPF₆ (Fluka), spectral grade acetonitrile and toluene (Burdick and Jackson), high purity dimethylaniline DMA (Aldrich), triflic acid (Sigma-Aldrich) and size exclusion Sephadex LH-20 gel (Sigma-Aldrich) were used as received.

[{(bpy)₂Ru(dpp)}₂Ru(dpq)](PF₆)₆ was synthesized by modification of previously reported method.²⁰ The terminal [(bpy)₂Ru(dpp)](PF₆)₂^{21,22} light absorber is assembled first and then coupled to the central Ru to produce [{(bpy)₂Ru(dpp)}₂RuCl₂](PF₆)₄.²³ The dichloro precursor [{(bpy)₂Ru(dpp)}₂RuCl₂](PF₆)₄ (244 mg, 0.168 mmol) and AgSO₃CF₃ (187 mg, 0.921 mmol) were heated at reflux in 20 ml of 95% ethanol for 4 hrs, under Ar. In another flask, excess dpq (160 mg, 0.563 mmol) was heated at reflux in 20 ml of ethylene glycol. After flash precipitation to remove the AgCl, the dichloro precursor solution was added in three aliquots to the refluxing dpq solution to maintain an excess of dpq during the reaction, favoring trimetallic formation. This mixture was heated to reflux for 24 hrs. The reaction mixture was cooled to room temperature and added to 500 ml of saturated aqueous KPF₆ to induce precipitation. The crude product was collected by vacuum filtration, dissolved in minimal acetonitrile, and reprecipitated in diethyl ether. Purification was achieved by size exclusion chromatography using Sephadex LH-20 column developed with 2:1/v:v ethanol:acetonitrile solvent mixture. The very first portion of color band is discarded and the major purple red band is collected. The product is flash precipitated from diethyl ether and dried under vacuum. Yield: 378 mg, 88%. FAB-MS: m/z [relatively abundance, ion]: 2403 [31, (M-PF₆-2H)⁺]; 2258 [100, (M-2 PF₆-2H)⁺]; 2117 [75, (M-3 PF₆+2H)⁺]; 1973 [22, (M-4PF₆+3H)⁺].

[{(bpy)₂Ru(dpp)}₂Ru(dpq)PtCl₂](PF₆)₆ was synthesized by reacting trimetallic complex [{(bpy)₂Ru(dpp)}₂Ru(dpq)](PF₆)₆ with [PtCl₂(DMSO)₂]¹⁹ [{(bpy)₂Ru(dpp)}₂Ru(dpq)](PF₆)₆ (147 mg, 0.058 mmol) and Pt(DMSO)₂Cl₂ (39 mg, 0.086 mmol) are mixed in 50 ml 95% ethanol and heated at reflux for 40 hours. Addition of saturated KPF₆ solution induces precipitation and the product is collected by vacuum filtration. The product is dissolved in a minimal amount of acetonitrile and flash precipitated in diethyl ether, collected by vacuum filtration and dried by rinsing with diethyl ether. Yield: 156 mg, 96%. Further purification is achieved by recrystallization from hot ethanol. Fast atom bombardment mass spectroscopy (FAB-MS) were performed by M-Scan Inc. at West Chester, Pa. The FAB-MS is performed on a VG Analytical ZAB 2-SE high field mass spectrometer using m-nitrobenzyl alcohol as a matrix.

FAB-MS results: m/z [relatively abundance, ion]: 2673 [17, (M-PF₆+H)⁺]; 2527 [33, (M-2 PF₆)⁺]; 2405 [82, (M-PF₆—PtCl₂)⁺]; 2260 [100, (M-2 PF₆—PtCl₂)⁺]; 2116 [33, (M-3 PF₆—PtCl₂)⁺]; 2055 [7, (M-PF₆-4bpy+8H)⁺].

### Example 2

### Electrochemical Properties of [{(bpy)₂Ru(dpp)}₂Ru(dpq)PtCl₂](PF₆)₆

Cyclic and square wave voltammetric experiments were conducted using a one-compartment, three-electrode cell, controlled with a BioAnalytical Systems (BAS) potentiostat. The working electrode was a 1.9 mm diameter glassy carbon disk, the auxiliary electrode a platinum wire and the reference electrode a Ag/AgCl electrode (0.29 V vs NHE), which was calibrated against the FeCp₂/FeCp₂⁺ redox couple (0.67 V vs NHE).²⁴ The supporting electrolyte was 0.1 M BU₄NPF₆, and the measurements were carried out in Burdick and Jackson spectro-grade acetonitrile under argon. Cyclic voltammetric measurements were made at a scan rate of 200 mV/s and square wave voltammetry at a scan rate of 100 mV/s. The working electrode was manually cleaned prior to each analysis.

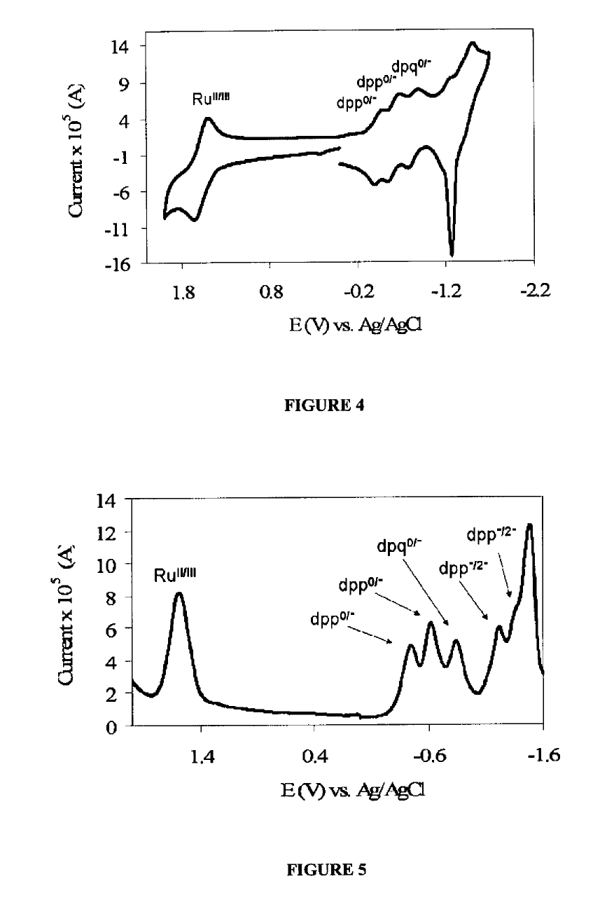

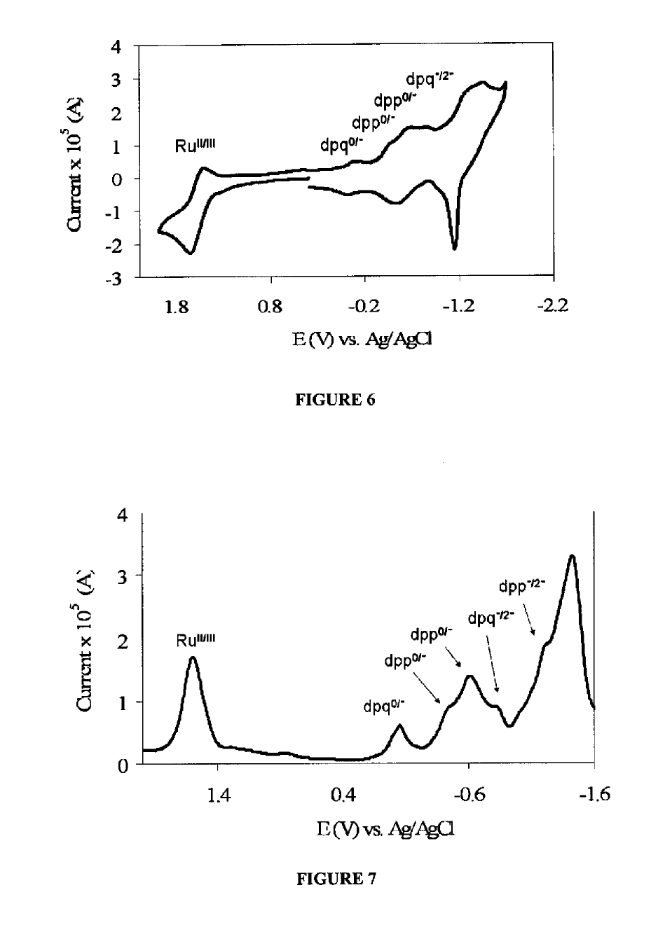

Cyclic and square wave voltammetric experiments were performed on both trimetallic [{(bpy)₂Ru(dpp)}₂Ru(dpq)](PF₆)₆ and tetrametallic [{(bpy)₂Ru(dpp)}₂Ru(dpq)PtCl₂](PF₆)₆ complexes. See FIGS. 4-7.

The electrochemical properties of [{(bpy)₂Ru(dpp)}₂Ru(dpq)PtCl₂](PF₆)₆ reflect its subunit components, as shown in Table 1. The complex displays an oxidative couple at 1.59 V vs. Ag/AgCl that represents the two terminal Ru^{II/III} processes. The first reduction at −0.08 V is assigned to the dpq^{0/-} couple consistent with the stabilized π* orbital of the dpq upon coordination to Ru^{II} and Pt^{II} and is followed by two dpp^{0/-} reductions. These properties predict a lowest lying charge separated (CS) state with an oxidized terminal Ru metal and a reduced dpq ligand with the promoted electron localized on the dpq ligand bound to Pt allowing for facile transfer to substrates.

TABLE 1
Redox properties of [{(bpy)₂Ru(dpp)}₂Ru(dpq)PtCl₂](PF₆)₆ and related
systems.^{a,b}

| | E_{1/2} (V) | | |
|---|---|---|---|
| Complex | Oxidations | Reductions | |
| [(bpy)₂Ru(dpq)PtCl₂](PF₆)₂ | 1.49 Pt^{II/III} | −0.30 | dpq^{0/—} |
| | 1.64 Ru^{II/III} | −0.99 | dpq^{—/2-} |
| [{(bpy)₂Ru(dpp)}₂Ru(dpq)](PF₆)₆ | 1.58 Ru^{II/III} | −0.42 | dpp^{0/—} |
| | | −0.62 | dpp^{0/—} |
| | | −0.82 | dpq^{0/—} |
| [{(bpy)₂Ru(dpp)}₂Ru(dpq)PtCl₂](PF₆)₆ | 1.59 Ru^{II/III} | −0.08 | dpq^{0/—} |
| | | −0.45 | dpp^{0/—} |
| | | −0.62 | dpp^{0/—} |

^{a}Data measured in 0.1 M Bu₄NPF₆ acetonitrile, E vs. Ag/AgCl
^{b}bpy = 2,2′-bipyridine, dpp = 2,3-bis(2-pyridyl)pyrazine and dpq = 2,3-bis(2-pyridyl)quinoxaline.

### Example 3

### Spectroelectrochemistry and Photoreduction of [{(bpy)₂Ru(dpp)}₂Ru(dpq)PtCl₂](PF₆)₆

Electronic Absorption Spectroscopy. Electronic absorption spectra were recorded at room temperature using a Hewlett-Packard 8452 diode array spectrophotometer with 2 nm resolution. Data was collected in 1 cm quartz cuvette after dissolving in UV-grade acetonitrile from Burdick and Jackson and extinction coefficients are the average of three measurements.

Spectroelectrochemistry and Photoreduction. Experiments were carried out to study the product of multiple electron reduction of [{(bpy)₂Ru(dpp)}₂Ru(dpq)PtCl₂](PF₆). The complex was reduced either electrochemically or photochemically using the sacrificial electron donor dimethylaniline (DMA).

Spectroelectrochemistry was performed using a home built three electrode thin cell. The cell consisted of two quartz plates separated by Teflon spacers to give a pathlength of ˜1.25 mm sealed using Parafilm. The electrodes consisted of a gold mesh working electrode, Ag wire pseudo reference electrode (−515 mV vs. Fc/Fc⁺) and carbon cloth auxiliary electrode. The cell was divided into auxiliary and working compartments with a tightly rolled VWR chemical wiper. Sample solutions were made to be 0.1 M NBu₄ PF₆ in CH₃CN and have an absorbance at 540 nm between 0.2 and 0.4. Potential was applied using the controlled-potential electrolysis function of a BioAnalytical Systems, Epsilon potentiostat. Short intervals of successively negative voltages were applied. Minor changes are observed in the electronic absorption spectrum at −0.20 V vs. Ag/AgCl, just negative of the dp^{0/-} couple. Significant changes were observed at an applied potential of −0.55 V, just negative of the first dpp^{0/-} couple. Reversal of the spectroelectrochemistry was performed by applying a potential of +1000 mV immediately following −0.55 V reduction.

Experiments were performed to compare the electrochemically reduced product spectrum to that of the photoreduced product. An acetonitrile solution of the complex was photolyzed with 455 nm LED light in the presence 5 mM DMA under argon atmosphere. A spectroscopic shift was observed that was nearly identical to that observed during spectroelectrochemistry at an applied voltage of −0.55 V.

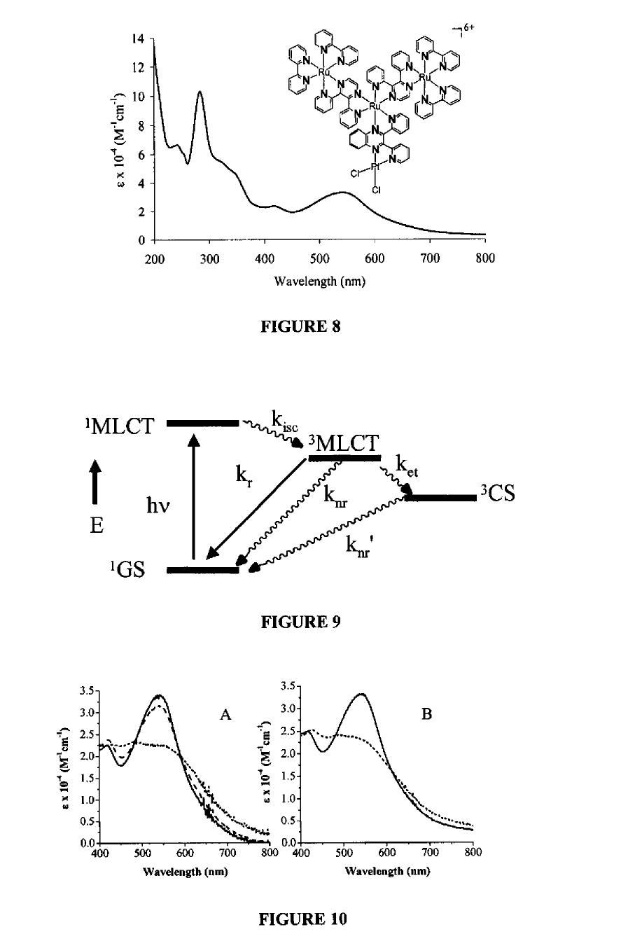

The light absorbing properties of the title complex are consistent with the sum of its chromophoric components. The electronic absorption spectrum of [{(bpy)₂Ru(dpp)}₂Ru(dpq)PtCl₂](PF₆)₆ in CH₃CN is illustrated in FIG. 8. The complex displays spectroscopy characteristic of Ru polyazine complexes and is quite similar to the [{(bpy)₂Ru(dpp)}₂Ru(dpq)](PF₆)₆ synthon. The UV region displays bpy (290 nm), dpp (320 nm) and dpq (360 nm) based π→π* transitions with the dpp and dpq based transitions occurring as a low energy shoulder on the more intense bpy peaks. The visible region of the spectrum is dominated by Ru based MLCT transitions with extinction coefficient at 542 nm of 33,200 M⁻¹ cm⁻¹, consistent with the number of overlapping transitions in this region. The Ru(dπ)→bpy(π*) CT transition occurs at 416 nm and peaks at ca. 500-580 nm correspond to the Ru(dπ)→μ-dpp(π*) and Ru(dπ)→μ-dpq(π*) CT transitions.

We have probed the photophysics of [{(bpy)₂Ru(dpp)}₂Ru(dpq)PtCl₂](PF₆)₆ and the related trimetallic synthon [{(bpy)₂Ru(dpp)}₂Ru(dpq)](PF₆)₆ and both display emissions from the terminal Ru(dπ)→dpp(71*) ³MLCT state. The trimetallic synthon emits at 745 nm with a lifetime of 133 ns and a quantum efficiency of 6.0×10⁻⁴ in RT CH₃CN solution. The lifetime of this emission is extended to 1.7 μs in a 4:1 EtOH/MeOH glass at 77 K. The title [{(bpy)₂Ru(dpp)}₂Ru(dpq)PtCl₂](PF₆)₆ emits at 745 nm with a lifetime of 92 ns and a quantum efficiency of 2.5×10⁻⁴ in RT CH₃CN solution. The lifetime is extended to 1.7 μs in 77 K 4:1 EtOH/MeOH glass. The reduction in emission of the tetrametallic relative to the trimetallic synthon is characteristic of an intramolecular quenching pathway with kₑₜ=3.4×10⁶ s⁻¹, FIG. 9. The equivalent emission lifetimes in 77 K in frozen glass result due to impeded electron transfer in a solid matrix, supporting population of the CS state in the complex [{(bpy)₂Ru(dpp)}₂Ru(dpq)PtCl₂](PF₆)₆ at RT.

The emission of the title complex [{(bpy)₂Ru(dpp)}₂Ru(dpq)PtCl₂](PF₆)₆ is quenched by addition of the electron donor dimethylaniline (DMA). The Stem Volmer analysis of the quenching of this ³MLCT emission by DMA is given in the supporting information. The data is fit to

Φₒ^{em}/Φ^{em}=1+tₒk_{q}[DMA]

yielding a k_{q} of 5.4×10⁹ M⁻¹s⁻¹, illustrative of the efficient quenching of the ³MLCT state by DMA.

Spectroelectrochemistry and photoreduction experiments were performed to compare the electrochemically reduced complex to the photoreduction product. The electrochemically reduced product was generated by electrolyzing an acetonitrile solution of [{(bpy)₂Ru(dpp)}₂Ru(dpq)PtCl₂](PF₆)₆ and 0.1 M NBu₄ PF₆ just negative of the dpq^{0/-} couple (−0.20 V vs Ag/AgCl) and the first dpp^{0/-} couple (−0.55 V). Little spectroscopic shift is observed following the first single electron reduction, implying that the central Ru(dπ)→μ-dpq(π*) transition is underlying other more intense transitions, FIG. 10A. Reduction by a second electron results in a significant decrease in the lowest energy MLCT band and rise of a shoulder at ˜650 nm. The observed spectroscopic shift upon reduction is consistent with previously reported changes to spectra of complexes containing two Ru(II)-centers bridged by dpp.²⁶ Photolysis of the complex in deoxygenated CH₃CN in the presence of dimethylaniline (DMA) results in a photoproduct with nearly identical spectroscopic signatures to the electrochemically generated product, FIG. 10B, illustrating that the photoreduction product has an electron localized on both the dpq, and at least one dpp bridge. This photoinitiated electron collection by [({(bpy)₂Ru(dpp)}Ru(dpq)PtCl₂](PF₆)₆ to generate the two electron reduced product is significant, representing one of only a handful of known photoinitiated electron collectors.

### Example 4

### Photocatalysis of Water to H₂

Photocatalysis. The photocatalytic production of hydrogen from water was assayed using light delivered from a LED array built locally.²⁷ The specific LED chosen is the Luxeon V star green 5W LED with dominant wavelength at 530 nm and 35 nm half-width. Samples were dissolved in spectrograde acetonitrile and then aqueous triflic acid CF₃SO₃H (pH=2.0) and DMA are added. The solutions are prepared in septum topped vials with flat, optical quality bottoms. All samples were deoxygenated with argon prior to photolysis and maintained under argon environment during photolysis. When photolyzed, the vials were placed in the instrumental compartments and irradiated from the bottom. The light intensity of LED (6.1×10¹⁸ photons/min) was determined by both chemical actinometry and calorimetry.

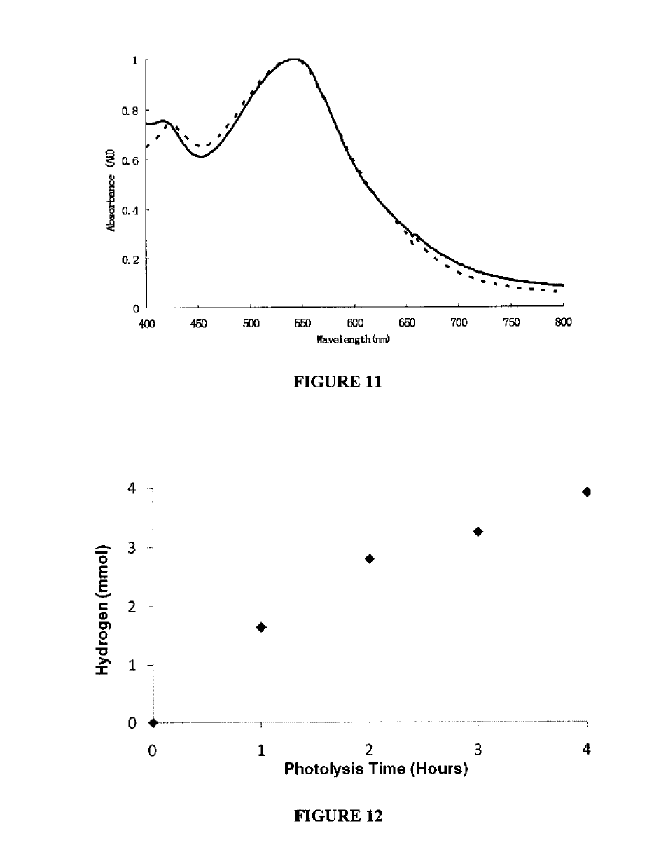

The conditions used here were: 1.5 mL metal complex stock solution is added to 0.40 mL DMA, 0.50 mL H₂O acidified to pH=2.0 with CF₃SO₃H, and 1.00 mL acetonitrile. The Ar head space was 2.10 mL. The final concentration for each component was: ca. 2.8×10⁻⁵ M metal complex, 0.92 M DMA, 1.5×10⁻³ M CF₃SO₃H, and 8.2×10⁻³ M H₂O. Assuming the pKa of protonated DMA is the same under our conditions as aqueous conditions to effective pH of our photolysis solutions are ca. 8.0. The catalyst [{(bpy)₂Ru(dpp)}₂Ru(dpq)PtCl₂](PF₆)₆ was stable in terms of photolysis during the experimental period and the comparison of electronic absorption spectra prior to and after the photolysis is shown in FIG. 11.

For the control experiments all conditions were kept the same except for the substitution of the tetrametallic catalyst by the indicated materials. For the Ru₃ ([{(bpy)₂Ru(dPp)}₂Ru(dpq)](PF₆)₆) and Pt colloid system, the conditions were kept the same as Ru₃Pt ([{(bpy)₂Ru(dpp)}₂Ru(dpq)PtCl₂](PF₆)₆) with equimolar Ru₃ and Pt colloid used in place of the tetrametallic Ru₃Pt catalyst. The Pt colloid stock suspension solution was prepared by mixing 18 mg commercial Pt colloid (10% weight) into 11 mL CH₃CN to make an 8.4×10⁻³ mol/L solution. For the control experiment by mixing Ru₃ and [PtCl₂(DMSO)₂] a stoichiometric amount of [PtCl₂(DMSO)₂] was used and other conditions were maintained as those for Ru₃Pt. The Hg experiments used a ca. 300 fold excess of Hg(l).

After photolysis, a 100 μL gas sample removed by syringe from the head space of the cell and injected into a GC for hydrogen analysis. The amount of hydrogen was determined by referring to a calibration curve. To account for the hydrogen dissolved in the solution, Henry's Law (equation 1) was used. The mole fraction of hydrogen at 1 atm was from the literature χ_{H2}1.78×10⁻⁴ and correlated well with our own measurement in CH₃CN.²⁸ P_{H2} is the partial pressure of hydrogen in the photolysis cell headspace, Pₛₐₜ is the partial pressure of hydrogen in the hydrogen saturation experiment, K_{H} is Henry's law constant, χ_{H2} is the mole fraction of hydrogen in the photolysis cell solution, and χₛₐₜ is the mole fraction of hydrogen in the hydrogen saturation experiment. The amount of hydrogen in the photolysis solution is then determined according to equation 3 where nₛₒₗ is the number of moles of solution, and n_{H2} is the amount of moles of hydrogen in the solution.

$$\begin{matrix}\frac{{P}_{{H}_{2}}}{{P}_{sat}} = \frac{( {K}_{H} ) ( {ϰ}_{{H}_{2}} )}{( {K}_{H} ) ( {ϰ}_{sat} )} & ( 1 ) \\ ϰ {H}_{2} = \frac{( {P}_{{H}_{2}} ) ( {ϰ}_{sat} )}{( {P}_{sat} )} = ( {P}_{{H}_{2}} ) ( {ϰ }_{sat} ) & ( 2 ) \\ {n}_{{H}_{2}} = ( {n}_{sol} ) ( {ϰ}_{{H}_{2}} ) & ( 3 )\end{matrix}$$

The total volume of hydrogen produced in a photolysis experiment is then the sum of the hydrogen found in the headspace as well as the hydrogen found in the solution. Typically the contribution of hydrogen in solution amounts to 10% of the total volume of hydrogen. The plot of hydrogen amount vs. photolysis time is shown in FIG. 12, in which each data point was the average of 2 sampling results. Full results are shown in Table 2.

TABLE 2
Photocatalytic Studies for H₂ Production Using
[{(bpy)₂Ru(dpp)}₂Ru(dpq)PtCl₂](PF₆)₆ and Related Controls. ^{a}

| | H₂O | DMA | Time | H₂ |
|---|---|---|---|---|
| Complex | (mL) | (mL) | (hr) | (μmol) |
| Ru₃Pt ^{b} | 0.50 | 0.40 | 1 | 1.6 |
| | 0.50 | 0.40 | 2 | 2.8 |
| | 0.50 | 0.40 | 3 | 3.3 |
| | 0.50 | 0.40 | 4 | 3.9 |
| | 0.50 | 0 | 3 | <0.01 |
| Ru₃ ^{c} | 0.50 | 0.40 | 3 | <0.01 |
| Ru₃ + | 0.50 | 0.40 | 3 | <0.01 |
| Pt(DMSO)₂Cl₂ | | | | |
| Ru₃ + Pt^{d} | 0.50 | 0.40 | 3 | 1.8 |
| Ru₃ + Pt^{d} + Hg | 0.50 | 0.40 | 3 | <0.01 |
| Ru₃Pt + Hg | 0.50 | 0.40 | 3 | 2.5 |

^{a} Photochemical generation of H₂ using 28 μM metal complex, 8.2 M H₂O, 1.5 mM added CF₃SO₃H with 0.92 M dimethylaniline (DMA), photolyzed for time indicated with a 530 nm 5 watt LED with 6.1 × 10¹⁸ photons/min, bpy = 2,2′-bipyridine, dpp = 2,3-bis(2-pyridyl)pyrazine and dpq = 2,3-bis(2-pyridyl)quinoxaline. Ref 13 describes LED construction.
^{b} Ru₃Pt = [{(bpy)₂Ru(dpp)}₂Ru(dpq)PtCl₂](PF₆)₆
^{c} Ru₃ = [{(bpy)₂Ru(dpp)}₂Ru(dpq)](PF₆)₆
^{d} Ru₃ + Pt = equimolar amounts of [{(bpy)₂Ru(dpp)}₂Ru(dpq)](PF₆)₆ and colloidal Pt

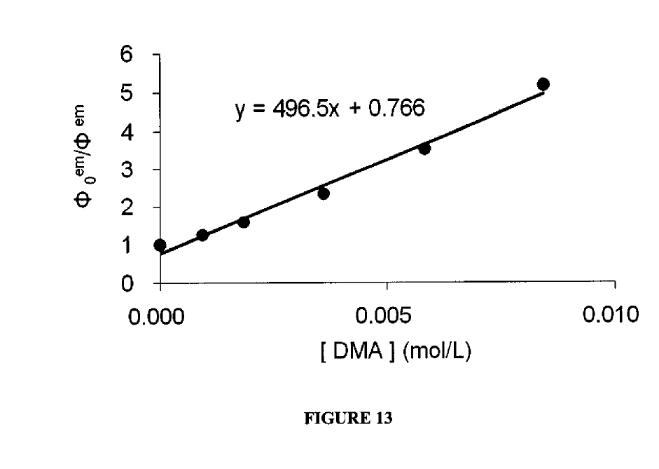

Stern-Volmer Plot (DMA Emission Quenching). The DMA emission quenching experiments on [{(bpy)₂Ru(dpp)}₂Ru(dpq)PtCl₂](PF₆)₆ were performed varying the concentration of DMA and studying the impact on emission quantum efficiency where Φ₀^{em}/Φ^{em} is the ratio of the quantum yield of emission in the absence and presence of the DMA quencher. The resultant Stern-Volmer plot is shown in FIG. 13.

The Stern-Volmer relation may also be written as:

Φₒ^{em}/Φ^{em}=1+tₒk_{q}[DMA]

Where Φₒ^{em} and Φ^{em} are the quantum yields of metal complex in the absence and presence of DMA. A plot of Φₒ^{em}/Φ^{em} versus [DMA] is expected to be linear, with a slope:

Kₛᵥ=Σₒk_{q}

where Kₛᵥ is the Stem-Volmer rate constant.
Kₛᵥ=497; τₒ=92 ns; k_{q}=5.4×10⁹ M⁻¹ s⁻¹

The title [{(bpy)₂Ru(dpp)}₂Ru(dpq)PtCl₂](PF₆)₆ photochemically reduces H₂O to H₂ in mixed CH₃CN/H₂O solution with the sacrificial electron donor DMA. The photochemical results and experimental details are provided in supporting information.

The title supramolecular assembly produced H₂ from H₂O when irradiated with 530 nm centered light from a 5W LED in the presence of DMA. After 4 hrs of photolysis 3.9 μmol of H₂ are produced representing 40 turnovers of the tetrametallic catalyst (3.3 μmol of H₂ produced in 3 hr). Systems with coordinated Pt^{II}, under photolytic conditions, could undergo to decomplexation of Pt^{II} and/or formation of Pt⁰ colioid. Assaying the formation of Pt(s), the addition of excess Hg to the photolysis solution above reduces H₂ production to 2.5 μmol in 3 hr. The photolysis of the trimetallic synthon [{(bpy)₂Ru(dpp)}Ru(dpq)]⁶⁺ alone or with [Pt(DMSO)₂Cl₂] does not lead to detectable H₂ production, indicative of the necessity of tetrametallic assembly to provide photocatalytic function. Photolysis of the synthon [{(bpy)₂Ru(dpp)}Ru(dpq)]⁶⁺ with equimolar Pt(s) produces 1.8 μmol of H₂ in 3 hr. Interestingly, the addition of Hg to this system results in the absence of detectable H₂, confirming the ability of Hg to scavenge Pt(s) in our system. These results together suggest that the tetrametallic assembly produces a catalytic system that is not recreated with the trimetallic synthon alone or with added [Pt(DMSO)₂Cl₂]. The results further demonstrate that the tetrametallic system functions more efficiently than the trimetallic synthons with added Pt(s). The addition of Hg to these photolysis systems suggest H₂ production by the tetrametallic complex may occur through two competing pathways. The functioning of this catalytic system provides longer term stability and higher turn over rates relative to related Ru,Pt systems.¹³

The complex [{(bpy)₂Ru(dpp)}Ru(dpq)PtCl₂](PF₆)₆ was synthesized and shown to be an efficient light absorber with a lowest lying CS state and shown to act as a multiple electron collector. The study of this complex shows the HOMO and LUMO are spatially separated terminal Ru and dpq localized, respectively. The complex absorbs strongly in the visible with overlapping Ru(dπ)→BL(π*) CT transitions. [{(bpy)₂Ru(dpp)}Ru(dpq)PtCl₂](PF₆)₆ and the Pt-free synthon [{(bpy)₂Ru(dpp)}Ru(dpq)](PF₆)₆ are weakly emissive, with the title complex having a lower RT emission quantum yield and shorter exited state lifetime. In alcoholic glass both have similar photophysical properties, indicating impeded electron transfer quenching. The results support formation of a terminal Ru(dπ)-dpq(π*) CS state in [{(bpy)₂Ru(dpp)}Ru(dpq)PtCl₂](PF₆)₆ following Ru(dπ)→dpp(π*) CT excitation. Spectroelectrochemical and photoreduction experiments support multielectron collection by this system. These experiments suggest the utility of [{(bpy)₂Ru(dpp)}Ru(dpq)PtCl₂](PF₆)₆ as a solar H₂ catalyst.

### REFERENCES

(1) (a) Campagna, S.; Serroni, S.; F. Puntoriero, F. Di Pietro, C.; Ricevuto, V. in Electron Transfer in Chemistry, Vol. 5, Balzani, V. Ed., 2001, 168 and references therein (b) Balzani, V.; Moggi, L.; Manfrin, M. F.; Bolletta, F.; Laurence, G. A, Coord. Chem. Rev. 1975, 15,321,
(2) Meyer, T. J. Pure Appl. Chem. 1986, 58, 1576.
(3) Kalyanasundaram, K. Coord. Chem. Rev. 1982, 46, 159.
(4) (a) Sutin, N.; Creutz, C. Pure Appl. Chem. 1980, 5Z 2717. (b) Brown, G. M.; Chan, S. F.; Creutz, C.; Schwarz, H. A.; Sutin, N. J. Am. Chem. Soc. 1979, 101, 7638. (c) Creutz, C.; Keller, A. D.; Sutin, N.; Zipp, A. P. J. Am. Chem. Soc. 1982, 104, 3618. (d) Chan, S.-F.; Chou, M.; Creutz, C.; Matsubura, T.; Sutin, N. J. Am. Chem. Soc. 1981, 103, 369.
(5) Du, P. W.; Schneider, J.; Jarosz, P.; Eisenberg, R. J. Am. Chem. Soc. 2006, 128, 7726.
(6) (a) Heyduk, A. F.; Nocera, D. G. Science 2001, 293, 1639. (b) Nocera, D.; Heyduk, A. PCT Int Appl. 2003, 157. (c) Gray, T.; Nocera, D. Chem. Comm. 2005, 12, 1540. (d) Esswein, A.; Veige, A.; Nocera, D. J. Am. Chem. Soc. 2005, 127(47), 16641.
(7) Balzani, V.; Juris, A.; Venturi, M.; Campagna, S.; Serroni, S. Chem. Rev. 1996, 96, 759 and references therein.
(8) Williams, R. L.; Toft, H. N.; Winkel, B.; Brewer, K. J. Inorg. Chem. 2003, 42, 4394.
(9) Yam, V. W-W.; Lee, V. W. M.; Cheung, K. K. J. Chem. Soc., Chem. Commun. 1994, 18, 2057.
(10) Milkevitch, M.; Brauns, E.; Brewer, K. J. Inorg. Chem. 1996, 35, 1737.
(11) Miao, R.; Zigler, D. F.; Brewer, K. J. Inorg. Chem. 2006, 45, 10413.
(12) Ozawa, H.; Haga, M.; Sakai, K. J. Am. Chem. Soc. 2006, 128, 4926.
(13) Rau, S.; Schafer, B.; Gleich, D.; Anders, E.; Rudolph, M.; Friedrich, M.; Görls, H.; Henry, W.; Vos, J. O. Angew. Chem. Int. Ed. 2006, 45, 6215.
(14) Lei, P.; Hedlund, M.; Lomoth, R.; Rensmo, H.; Johansson, O.; Hammarstrom, L. J. Am. Chem. Soc. 2008, 130,26
(15) Photochemical production of hydrogen and oxygen from water: A review and state of the art,” Edmond Amouyal Solar Energy Materials and Solar Cells 1995, 38, 249.
(16) Hydrogen Generation by Visible Light Irradiation of Aqueous Solutions of Metal Complexes. An approach to the Photochemical Conversion and Storage of Solar Energy,” Michele Kirch, Jean-Marie Lehn, Jean-Pierre Sauvage, Helv. Chim. Acta 1979, 62, 1345).
(17) Goodwin, H. A.; Lions, F. J. Am. Chem. Soc. 1959, 81, 6415.
(18) Molnar, S. M.; Neville, K. R.; Jensen, G. E.; Brewer, K. J. Inorg. Chim. Acta 1993, 206, 69.
(19) Sahai, R.; Rillema, D. P. J. Chem. Soc., Chem. Commun. 1986, 1133.
(20) Brauns, E.; Jones, S. W.; Clark, J. A.; Molnar, S. M.; Kawanishi, Y.; Brewer, K. J. Inorg. Chem. 1997, 36, 2861.
(21) Fuchs, Y.; Lofters, S.; Dieter, T.; Shi, W.; Morgan, R.; Strekas, T. C.; Gafney, H. D.; Baker, A. D. J. Am. Chem. Soc. 1987, 109, 2691.
(22) Rillema, D. P.; Mack, K. B. Inorg. Chem. 1982, 21, 3849.
(23) Richter, M. M.; Brewer, K. J. Inorg. Chem. 1993, 32, 5762.
(24) Gennett, T.; Milner, D. F.; Weaver, M. J. J. Phys. Chem. 1985, 89, 2787.
(25) Brauns, E.; Jones, S. W.; Clark, J. A.; Molnar, S. M.; Kawanishi, Y.; Brewer, K. J. Inorg. Chem. 1997, 36,2861.
(26) Berger, R. M. Inorg. Chem. 1990, 29, 1920.
(27) Brown, J. R.; Elvington, M.; Mongelli, M. T.; Zigler, D. F.; Brewer, K. J. Proc. of SPIE 2006, 6340, 634017.
(28) Purwanto; Deshpande, R. M.; Chaudhari, R. V.; Delmas, H. J. Chem. Eng. Data 1996, 41, 1414.
(29) Sullivan, B. P.; Salmon, D. J.; Meyer, T. J. Inorg. Chem. 1978, 17, 3334.
(30) Braunstein, C. H.; Baker, A. D.; Strekas, T. C.; Gafney, H. D. Inorg. Chem. 1984, 23, 857.
(31) Puntoriero, F.; Serroni, S.; Galletta, M.; Juris, A.; Licciardello, A.; Chiorboli, C.; Campagna, S.; Scandola, F. Chem Phys Chem 2005, 6, 129.

What is claimed is:

1. A supramolecular complex for photocatalyzing the reduction of a substrate comprising:
a charge transfer light absorbing metal center;
an electron collector ligand; and
a catalytically active metal;
wherein the charge transfer light absorber transfers a charge to the electron collector ligand, and wherein when sufficient charge is accumulated on the electron collector ligand, the charge is transferred through the catalytically active metal to the substrate.

2. The supramolecular complex of claim 1, further comprising a terminal ligand.

3. The supramolecular complex of claim 3, wherein the charge transfer light absorber metal center comprises a metal selected from the group consisting of: ruthenium(II), osmium(II), rhenium (I), and iron(II).

4. The supramolecular complex of claim 1, wherein the catalytically active metal comprises a metal selected from the group consisting of: platinum (II), palladium (II), cobalt (I), rhodium (I), and iridium (I).

5. The supramolecular complex of claim 1, wherein the electron collector ligand is selected from the group consisting of: 2,2′-bipyridine, 1,10-phenanthroline, 2,2′:6′,2″-terpyridine, 2,3-bis(2-pyridyl)pyrazine, 2,2′-bipyridimidine, 2,3-bis(2-pyridyl)quinoxaline, 2,3-bis(2-pyrdiyl)benzoquinozline, and 2,3,5,6,-tetrakis(2-pyridyl)pyrazine.

6. The supramolecular complex of claim 2, wherein the terminal ligand is selected from the group consisting of: 2,2′-bipyridine, 2,2′:6′,2″-terpyridine, 2,3-bis(2-pyridyl)pyrazine, 2,2′-bipyridimidine, 2,3-bis(2-pyridyl)quinoxaline, 2,3,5,6,-tetrakis(2-pyridyl)pyrazine, 2,2′-phenylpyridine, triphenylphosphine, diethylphenylphosphine Cl⁻, Br⁻, I⁻, F⁻, CN, CO, COOH, and CH₃CN.

7. The supramolecular complex of claim 2, wherein the terminal ligand is selected from phosphines having the general formula PR₃; where each R may be the same or different and may be alkyl or aryl groups having between 1 and 12 carbons.

8. The supramolecular complex of claim 2, wherein the terminal ligand is selected from simple amines having a general formula NR₃; wherein each R may be the same or different and may be hydrogen or alkyl or aryl groups having between 1 and 12 carbons.

9. A supramolecular complex for photocatalyzing the reduction of a substrate of the formula [{(bpy)₂Ru(dpp)}₂Ru(dpq)PtCl₂]X₆; wherein X is a counterion.

10. The supramolecular complex of claim 9, wherein X is PF₆.

11. A method for photocatalytic reduction of a substrate comprising:
providing a substrate to be reduced;
contacting the substrate with a supramolecular complex comprising:
a charge transfer light absorbing metal center;
an electron collector ligand; and
a catalytically active metal; and
exposing the supramolecular complex to a source of radiant energy for a period of time suitable to cause reduction of the substrate.

12. The method for photocatalytic reduction of claim 11, wherein the catalytically active metal is selected from the group consisting of: platinum (II), palladium (II), cobalt (I), rhodium(I), and iridium (I).

13. The method for photocatalytic reduction of claim 11, wherein the substrate is water.

14. The method for photocatalytic reduction of claim 11, wherein the substrate is selected from the group consisting of carbon dioxide, carbon monoxide, methanol and nitrobenzene.

15. The method for photocatalytic reduction of claim 11, wherein the source of radiant energy is visible light.

16. The method for photocatalytic reduction of claim 11, wherein the source of radiant energy is ultraviolet light.

17. The method for photocatalytic reduction of claim 11, further comprising providing an electron donor prior to exposing the supramolecular complex to a source of radiant energy.

18. The method for photocatalytic reduction of claim 17, wherein the electron donor is selected from the group consisting of: dimethylaniline, triethanolamine, triethylamine, ethylenediamenetetraacetic acid and ascorbic acid.

19. A system for photocatalytic reduction of a substrate comprising:
a vessel containing the substrate and a supramolecular complex comprising:
a charge transfer light absorbing metal center;
an electron collector ligand; and
a catalytically active metal; and
a source of radiant energy;
wherein when the supramolecular complex is exposed to the radiant energy, the supramolecular complex catalyzes the reduction of the substrate.

20. The system for photocatalytic reduction of claim 19, wherein the catalytically active metal is selected from the group consisting of: platinum (II), palladium (II), cobalt (I), rhodium(I), and iridium (I).
